(19) United States
(12) Patent Application Publication
(10) Pub. No.: US 2019/0167195 A1
(43) Pub. Date: Jun. 6, 2019

(54) SYSTEMS AND METHODS FOR PERFORMING DIAGNOSTIC PROCEDURES FOR A VOLUME CLAMP FINGER CUFF

(71) Applicant: Edwards Lifesciences Corporation, Irvine, CA (US)

(72) Inventors: Blake W. Axelrod, Sierra Madre, CA (US); Alexander H. Siemons, Yorba Linda, CA (US); Virginia Qi Lin, Costa Mesa, CA (US); Leah Paige Gaffney, Irvine, CA (US); Laura J. Livant, Seal Beach, CA (US)

(21) Appl. No.: 16/179,643

(22) Filed: Nov. 2, 2018

Related U.S. Application Data
(60) Provisional application No. 62/594,111, filed on Dec. 4, 2017.

(51) Int. Cl. A61B 5/00; A61B 5/0225; A61B 5/022; A61B 5/021; A61B 5/0295; A61B 5/026

(52) U.S. Cl. CPC A61B 5/6843; A61B 5/02255; A61B 5/0261; A61B 5/02116; A61B 5/0295; A61B 5/02241

(57) ABSTRACT

Disclosed is a system to monitor a finger cuff connectable to a patient's finger to be used in measuring the patient's blood pressure by a blood pressure measurement system utilizing the volume clamp method and to measure the plethysmogram of the finger cuff. The system comprises the finger cuff that includes an enclosing portion that encloses a patient's finger. The enclosing portion includes a bladder and a light emitting diode (LED) and photodiode (PD) pair. The system further comprises a processor to: command applying pneumatic pressure to the bladder of the finger cuff from a low pressure to a high pressure; measure the plethysmogram of the finger cuff as the pressure increases from the low pressure to the high pressure; and determine fitness of the finger cuff on the patient's finger based on the measured plethysmogram. When the finger cuff is placed around the patient's finger, the bladder and the LED-PD pair aid the processor in measuring the plethysmogram.

### RELATED APPLICATION

This application claims priority to U.S. Provisional Application No. 62/594,111, filed Dec. 4, 2017, the contents of which are incorporated herein by reference in its entirety.

### BACKGROUND

### Field

Embodiments of the invention relate generally to non-invasive blood pressure measurement. More particularly, embodiments of the invention relate to the performance of diagnostic procedures for a volume clamp finger cuff.

### Relevant Background

Volume clamping is a technique for non-invasively measuring blood pressure in which pressure is applied to a patient's finger in such a manner that arterial pressure may be balanced by a time varying pressure to maintain a constant arterial volume. In a properly fitted and calibrated system, the applied time varying pressure is equal to the arterial blood pressure in the finger. The applied time varying pressure may be measured to provide a reading of the patient's arterial blood pressure.

This may be accomplished by a finger cuff that is arranged or wrapped around a finger of a patient. The finger cuff may include an infrared light source, an infrared sensor, and an inflatable bladder. The infrared light may be sent through the finger in which a finger artery is present. The infrared sensor picks up the infrared light and the amount of infrared light registered by the sensor may be inversely proportional to the artery diameter and indicative of the pressure in the artery.

In the finger cuff implementation, by inflating the bladder in the finger cuff, a pressure is exerted on the finger artery. If the pressure is high enough, it will compress the artery and the amount of light registered by the sensor will increase. The amount of pressure necessary in the inflatable bladder to compress the artery is dependent on the blood pressure. By controlling the pressure of the inflatable bladder such that the diameter of the finger artery is kept constant, the blood pressure may be monitored in very precise detail as the pressure in the inflatable bladder is directly linked to the blood pressure. In a typical present day finger cuff implementation, a volume clamp system is used with the finger cuff. The volume clamp system typically includes a pressure generating system and a regulating system that includes: a pump, a valve, and a pressure sensor in a closed loop feedback system that are used in the measurement of the arterial volume. To accurately measure blood pressure, the feedback loop provides sufficient pressure generating and releasing capabilities to match the pressure oscillations of the patient's blood pressure.

Today, finger cuff based blood pressure monitoring devices generally use the same technology (e.g., photoplethysmography or similar technologies) to measure blood pressure. Unfortunately, such finger cuff devices may not be easily attachable to a patient's finger and may not be that accurate due to the finger cuff's positioning on the patient's finger. That is, attaching the finger cuff in a suboptimal way may negatively influence the measurement reliability and accuracy of the volume clamp system. For example, a loose finger cuff on the patient's finger may require the bladder to stretch in order to reach the finger. Therefore, this may lead to the additional consumption of energy and a reading of an artificially high blood pressure.

### SUMMARY

Embodiments of the invention may relate to a system to monitor a finger cuff connectable to a patient's finger to be used in measuring the patient's blood pressure by a blood pressure measurement system utilizing the volume clamp method and to measure the plethysmogram of the finger cuff. The system comprises the finger cuff that includes an enclosing portion that encloses a patient's finger. The enclosing portion includes a bladder and a light emitting diode (LED) and photodiode (PD) pair. The system further comprises a processor to: command applying pneumatic pressure to the bladder of the finger cuff from a low pressure to a high pressure; measure the plethysmogram of the finger cuff as the pressure increases from the low pressure to the high pressure; and determine the fitness of the finger cuff on the patient's finger based on the measured plethysmogram. When the finger cuff is placed around the patient's finger, the bladder and the LED-PD pair aid the processor in measuring the plethysmogram.

### BRIEF DESCRIPTION OF THE DRAWINGS

FIG. 1 is a diagram of an example of a blood pressure measurement system according to one embodiment.

FIG. 2 is a block diagram illustrating a finger cuff and a pressure generating and regulating system.

FIGS. 3A-3C are diagrams illustrating the measured plethysmograms of a finger cuff according to embodiments of the invention.

FIGS. 4A-4C are diagrams illustrating additional measured plethysmograms of the finger cuff according to embodiments of the invention.

FIGS. 5A-5C are diagrams illustrating additional measured plethysmograms of the finger cuff according to embodiments of the invention.

FIG. 6 a flow diagram of a method for measuring the pulsatility of a finger cuff according to embodiments of the invention.

FIG. 7 is a flow diagram of a method for determining whether a finger cuff is properly fitted on a patient's finger according to embodiments of the invention.

FIG. 8 is a flow diagram of another method for determining whether the finger cuff is properly fitted on the patient's finger according to embodiments of the invention.

FIG. 9 is a block diagram illustrating example control circuitry.

### DETAILED DESCRIPTION

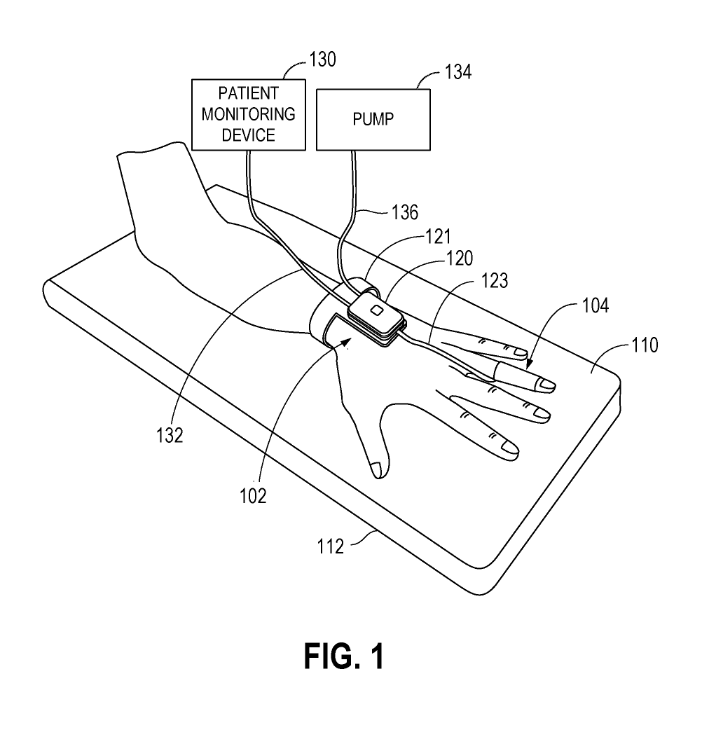

With reference to FIG. 1, which illustrates an example of a blood pressure measurement system according to one embodiment, a blood pressure measurement system 102 that includes a finger cuff 104 that may be attached to a patient's finger and a blood pressure measurement controller 120, which may be attached to the patient's body (e.g., a patient's wrist or hand) is shown.

The blood pressure measurement system 102 may further be connected to a patient monitoring device 130, and, in some embodiments, a pump 134. Further, finger cuff 104 may include a bladder (not shown) and an LED-PD pair (not shown), which are conventional for finger cuffs.

In one embodiment, the blood pressure measurement system 102 may include a pressure measurement controller 120 that includes: a small internal pump, a small internal valve, a pressure sensor, and control circuity. In this embodiment, the control circuitry may be configured to: control the pneumatic pressure applied by the internal pump to the bladder of the finger cuff 104 to replicate the patient's blood pressure based upon measuring the pleth signal received from the LED-PD pair of the finger cuff 104. Further, the control circuitry may be configured to: control the opening of the internal valve to release pneumatic pressure from the bladder; or the internal valve may simply be an orifice that is not controlled. Additionally, the control circuitry may be configured to: measure the patient's blood pressure by monitoring the pressure of the bladder based upon the input from a pressure sensor, which should be the same as patient's blood pressure, and may display the patient's blood pressure on the patient monitoring device 130.

In another embodiment, a conventional pressure generating and regulating system may be utilized, in which, a pump 134 is located remotely from the body of the patient. In this embodiment, the blood pressure measurement controller 120 receives pneumatic pressure from remote pump 134 through tube 136 and passes on the pneumatic pressure through tube 123 to the bladder of finger cuff 104. Blood pressure measurement device controller 120 may also control the pneumatic pressure (e.g., utilizing a controllable valve) applied to the finger cuff 104 as well as other functions. In this example, the pneumatic pressure applied by the pump 134 to the bladder of finger cuff 104 to replicate the patient's blood pressure based upon measuring the pleth signal received from the LED-PD pair of the finger cuff 104 (e.g., to keep the pleth signal constant) and measuring the patient's blood pressure by monitoring the pressure of the bladder may be controlled by the blood pressure measurement controller 120 and/or a remote computing device and/or the pump 134 and/or the patient monitoring device 130 to implement the volume clamping method. In some embodiments, a blood pressure measurement controller 120 is not used at all and there is simply a connection from tube 136 from a remote pump 134 including a remote pressure regulatory system to finger cuff 104, and all processing for the pressure generating and regulatory system, data processing, and display is performed by a remote computing device.

Continuing with this example, as shown in FIG. 1, a patient's hand may be placed on the face 110 of an arm rest 112 for measuring a patient's blood pressure with the blood pressure measurement system 102. The blood pressure measurement controller 120 of the blood pressure measurement system 102 may be coupled to a bladder of the finger cuff 104 in order to provide pneumatic pressure to the bladder for use in blood pressure measurement. Blood pressure measurement controller 120 may be coupled to the patient monitoring device 130 through a power/data cable 132. Also, in one embodiment, as previously described, in a remote implementation, blood pressure measurement controller 120 may be coupled to a remote pump 134 through tube 136 to receive pneumatic pressure for the bladder of the finger cuff 104. The patient monitoring device 130 may be any type of medical electronic device that may read, collect, process, display, etc., physiological readings/data of a patient including blood pressure, as well as any other suitable physiological patient readings. Accordingly, power/data cable 132 may transmit data to and from patient monitoring device 130 and also may provide power from the patient monitoring device 130 to the blood pressure measurement controller 120 and finger cuff 104.

As can be seen in FIG. 1, in one example, the finger cuff 104 may be attached to a patient's finger and the blood pressure measurement controller 120 may be attached on the patient's hand or wrist with an attachment bracelet 121 that wraps around the patient's wrist or hand. The attachment bracelet 121 may be metal, plastic, Velcro, etc. It should be appreciated that this is just one example of attaching a blood pressure measurement controller 120 and that any suitable way of attaching a blood pressure measurement controller to a patient's body or in close proximity to a patient's body may be utilized and that, in some embodiments, a blood pressure measurement controller 120 may not be used at all. It should further be appreciated that the finger cuff 104 may be connected to a blood pressure measurement controller described herein, or a pressure generating and regulating system of any other kind, such as a pressure generating and regulating system that is located remotely from the body of the patient. Any kind of pressure generating and regulating system can be used, including but not limited to the blood pressure measurement controller, and may be described simply as a pressure generating and regulating system that may be used with a finger cuff 104 including an LED-PD pair and a bladder to implement the volume clamping method.

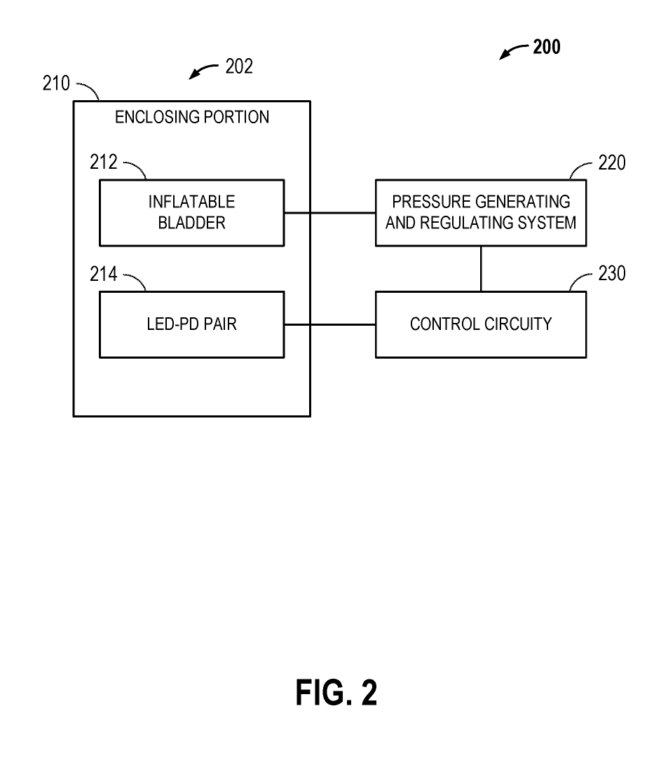

FIG. 2 is a block diagram illustrating a finger cuff and a pressure generating and regulating system. As an example, as shown in FIG. 2, finger cuff 202 may include an enclosing portion 210, an inflatable bladder 212 and an LED-PD pair 214. The enclosing portion 210 may encircle or enclose a patient's finger and include inflatable bladder 212 and LED-PD pair 214. The inflatable bladder 212 may be pneumatically connected to a pressure generating and regulating system 220. The LED may be used to illuminate the finger skin and light absorption or reflection may be detected with the PD. The pressure generating and regulating system 220 and control circuitry (e.g., including a processor) 230 may generate, measure, and regulate pneumatic pressure that inflates or deflates the inflatable bladder 212, and may further comprise such elements as a pump, a valve, a pressure sensor, and/or other suitable elements, as previously described. In particular, pressure generating and regulating system 220 in cooperation with control circuitry 230 may be configured to implement a volume clamp method with the finger cuff 202 by: applying pneumatic pressure to the inflatable bladder 212 of the finger cuff 202 to replicate the patient's blood pressure based upon measuring the pleth signal received from the LED-PD pair 214 of the finger cuff 202 (e.g., to keep the pleth signal constant); and measuring the patient's blood pressure by monitoring the pressure of the inflatable bladder 212 based upon input from a pressure sensor, which should be the same as patient's blood pressure, and may further command the display of the patient's blood pressure on the patient monitoring device.

In one embodiment, pressure generating and regulating system 220 and control circuitry 230 may automatically perform diagnostic procedures (e.g., a series of tests) to assess equipment statuses (e.g., pump performance, valve performance), finger cuff fitness (e.g., tightness, location and fit), and/or patient suitability (e.g., patient's perfusion) for the volume clamp method. In some embodiments, the diagnostic procedures may be performed at system start-up and/or during system run time of the pressure generating and regulating system 220 and/or control circuitry 230 to obtain and assess various metrics associated with the equipment statuses, finger cuff fitness, and patient suitability.

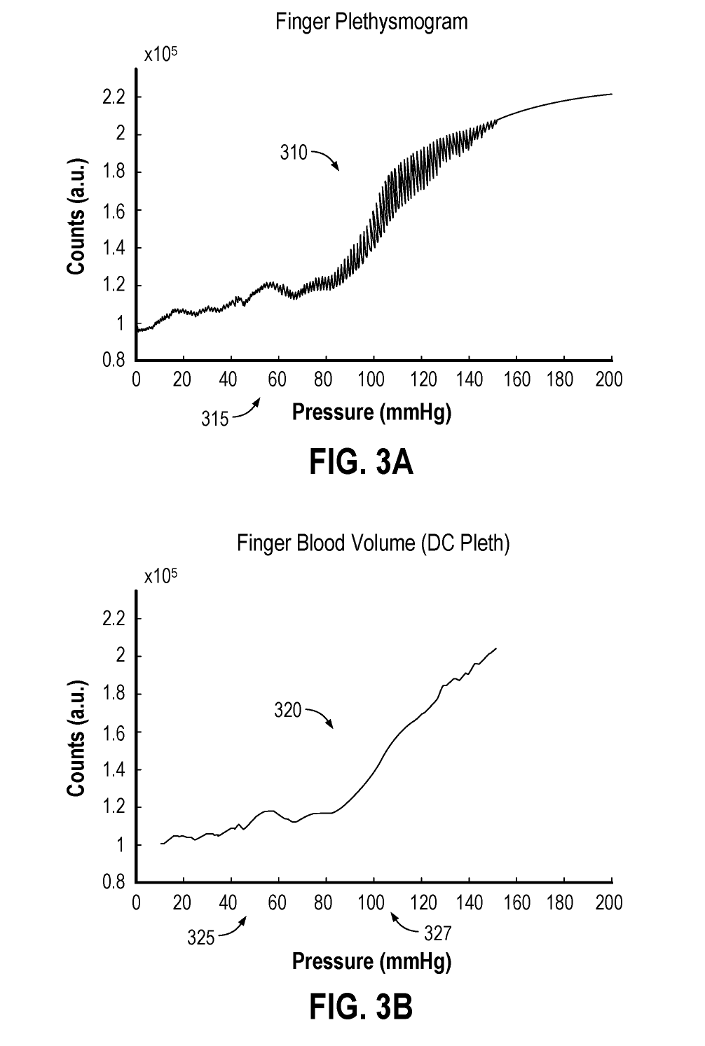

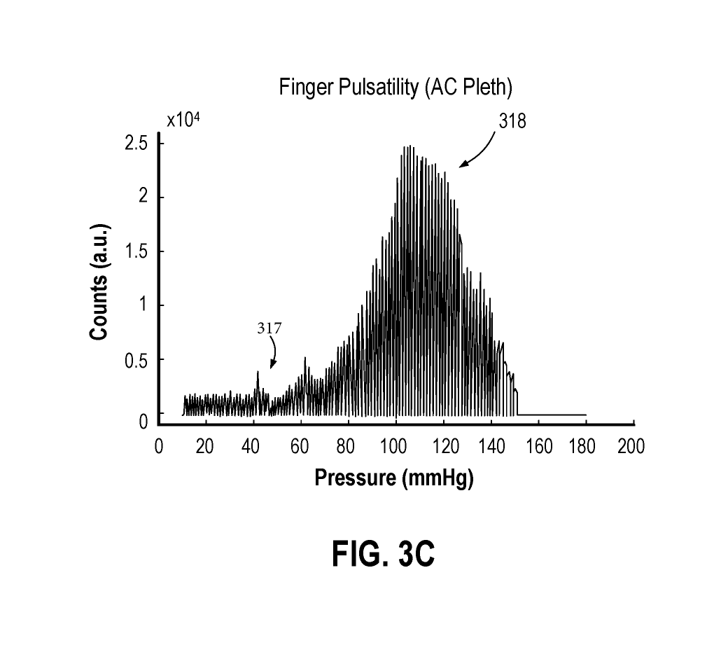

A plethysmogram, or pleth signal, obtained by the bladder 212 and LED-PD pair 214 contains two parts. The finger pulsatility, also known as the AC pleth, is the pulsation due to the subject's heart beats. The pulsatility can be changed by applying pressure to the finger, for example by the bladder 212, that confine the artery's movement within the finger. The finger blood volume, also known as the DC pleth, excludes changes due to the subject's heart beats. Rather, it is the steady background level of light absorbing blood and tissue in the finger. The finger blood volume can be changed by applying pressure to the finger, for example by the bladder 212, which squeezes blood, both arterial and venous, out of the finger. Both pulsatility and blood volume can be characterized as functions of external pressure applied by the bladder 212. FIG. 3A shows the plethysmogram as a function of pressure applied by a bladder, including both pulsatility and blood volume, for a typical finger. FIG. 3B separates out just the steady state blood volume from 3A, as pressure increases light absorbing blood is pushed out of the finger and the DC Pleth increases. FIG. 3C separates out just the pulsatility from 3A, at low pressures the artery is fully stretched by the subject's blood pressure resulting in low pulsatility, as the pressure increases the artery is compressed into a highly elastic state that yields large pulsations with each heartbeat, and at high pressure the artery is fully compressed and very little blood is able to enter the finger at each heartbeat. Thus both portions of the plethysmogram contain information relating to the interaction between subject's finger and the bladder.

In particular, pressure generating and regulating system 220 in cooperation with control circuitry 230 may apply pneumatic pressure to bladder 212 from a low pressure, e.g., 20-40 millimeter of mercury (mmHg), to a high pressure (e.g., 200 mmHg) and measure the plethysmogram of finger cuff 202 as the pressure increases from the low pressure to the high pressure. That is, in one embodiment, the pressure generating and regulating system 220 and control circuitry 230 (by way of bladder 212 and LED-PD pair 214) may make continuous volumetric measurements (or plethysmogram) of arterial blood flows within the patient's finger as the pressure increases from the low pressure to the high pressure. Thus, pulsatility and blood volume in the finger may be detected based on the plethysmogram, which may be generated based on the pleth signal received from the PD of LED-PD pair 214. Based on the measured pulsatility and/or blood volume of finger cuff 202, the control circuitry 230 may determine the fitness of finger cuff 202 on the patient's finger. For example, the control circuitry 230 may determine whether finger cuff 202 is loose, properly fitted, or too tight on the patient's finger.

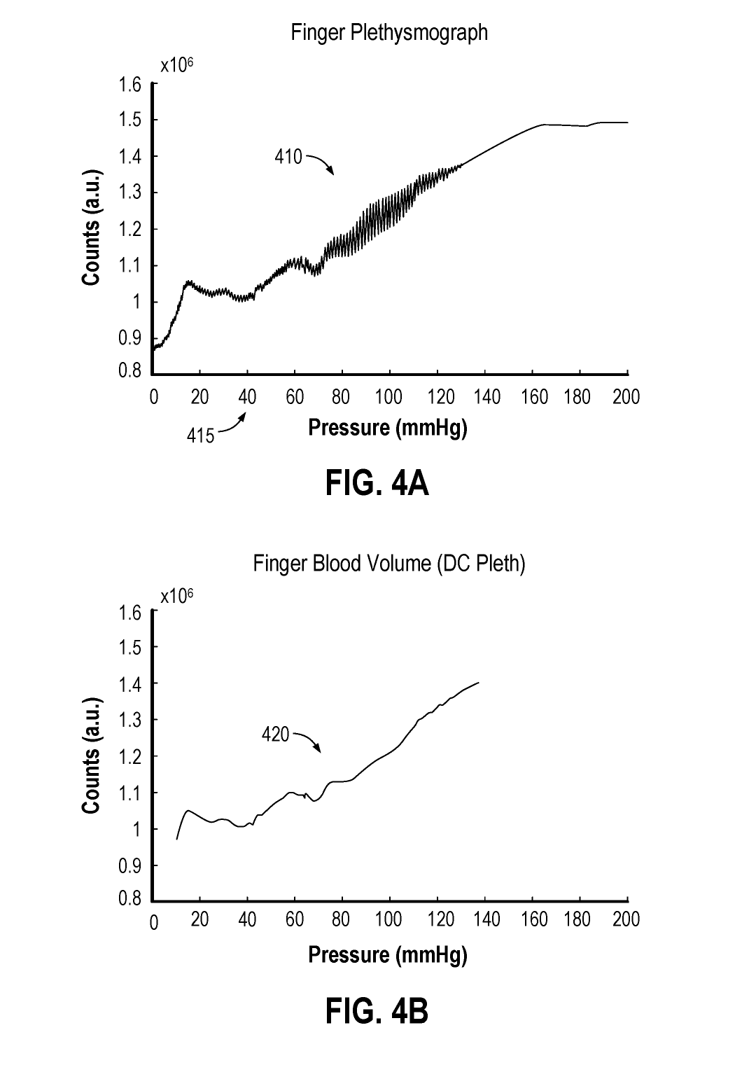

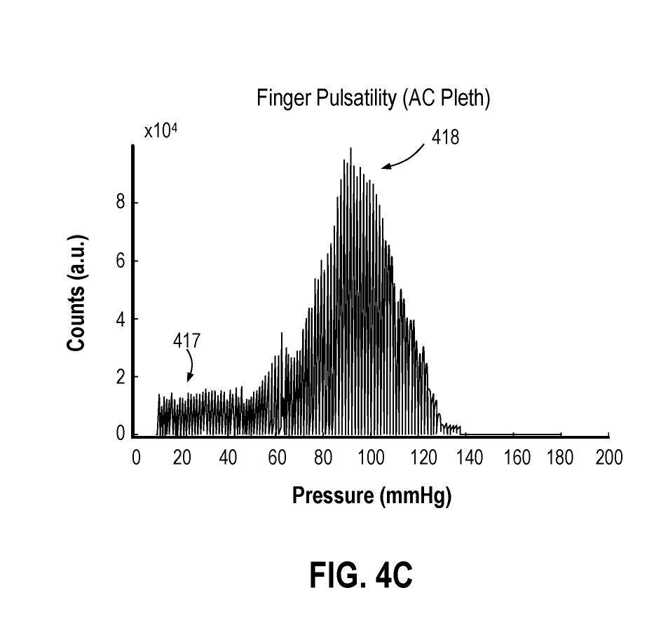

In some embodiments, in determining the fitness of finger cuff 202, the pressure generating and regulating system 220 and control circuitry 230 may apply multiple pressure sequences to the finger cuff 202, and the pleth signal received from LED-PD pair 214 may be acquired and analyzed. For example, a low pressure (e.g., 20-40 mmHg) may be applied to bladder 212, and the pleth signal may be measured as the pressure of the bladder increases to the low pressure. A high pressure (200 mmHg) may then be applied to bladder 212 and held for a time period (e.g., 1 second), and during such time period, the pleth signal may again be measured. Subsequently, the pressure from bladder 212 may be released (e.g., by turning off the pump) and the pressure decay may be observed. The pleth signal may be measured throughout the pressure decay, and in some embodiments, for an additional time period (e.g., 3 seconds or any suitable amount of time) after the pump has been turned off. Based on the various measurements of the pleth signal, as previously described, the control circuitry 230 may determine whether finger cuff 202 is loose, properly fitted, or too tight on the patient's finger (as described in more detail with respect to FIGS. 3A-3C, 4A-C and 5A-5C herein below). Further, based on the various measurements of the pleth signal, as previously described, the control circuitry 230 may perform various equipment status checks, as described below.

In some embodiments, with respect to equipment statuses, control circuitry 230 may check pump performance of the pressure generating and regulating system 220. For example, control circuitry 230 may control a designated pneumatic pressure applied by the pump to the bladder 212 of the finger cuff 202. Control circuitry 230 may then determine whether the pump has reached the designated pressure. If the pump does not reach the designated pressure, control circuitry 230 may determine that the pump is inoperable. Otherwise, control circuitry 230 may then determine whether the ratio of the designated pressure to the power of the pump during pressure impulse is within a desired ratio. If the ratio is not within the desired ratio, control circuitry 230 may determine that the pump is inoperable. In this case, an operator (e.g., healthcare provider) may be instructed to replace parts of the pump (e.g., servo unit).

In some embodiments, if a valve is present in pressure generating and regulating system 220, the valve may be utilized to release pneumatic pressure from bladder 212. In this case, control circuitry 230 may determine whether a leakage rate with the pump off and the valve closed is above a leakage threshold. If the leakage rate is not above the leakage threshold, control circuitry 230 may determine that a leakage exists in pressure generating and regulating system 220. In this scenario, the operator may be instructed to check one or more connections between the servo and finger cuff 202. If such condition occurs for a number of times (e.g., three times), the operator may be instructed to replace finger cuff 202. If the condition continues to occur after the replacement of finger cuff 202, control circuitry 230 may determine that the valve is inoperable and instruct the operator to replace, for example a servo unit associated with the valve.

In some embodiments, with respect to patient suitability, control circuitry 230 may check the patient's perfusion, which is the volume of blood flow through the finger. For example, control circuitry 230 may determine whether the blood volume measured at the end of recovery time, for example a DC Pleth magnitude, has returned to an initial value measured at the low pressure, thereby indicating that blood has returned to the finger. If the blood volume measured at the end of the recovery time has not returned to the initial value (i.e., the blood has not fully returned), control circuitry 230 may determine that the patient's perfusion is too low for the volume clamp system to operate properly. In this case, the operator may be instructed to increase the patient's perfusion by warming the hand or to select a different pressure monitoring technology.

Referring to FIGS. 3A-3C, diagrams illustrating measured plethysmogram of finger cuff 202 according to embodiments of the invention are shown. In some embodiments, FIGS. 3A-3C illustrate the plethysmogram and its components, the finger pulsatility and finger blood volume, obtained by pressure generating and regulating system 220 and control circuitry 230, as previously described, applying pressure to finger cuff 202 and measuring pleth signals. With reference to FIG. 3A, the diagram illustrates a gradual pressure ramp response, and particularly, an example of the changing pleth signal as a function of pressure. As shown in the diagram, a trace 310 shows the measured plethysmogram in arbitrary units (a.u.) that corresponds to the pneumatic pressures (which may be measured in mmHg) applied to the patient's finger.

As can be seen on FIGS. 3A-3C and 4A-4C, diagrams illustrating additional measured plethysmogram of finger cuff 202 according to embodiments of the invention are shown. With reference to FIG. 3A, the diagram illustrates a pressure ramp response, and particularly, an example of a changing pleth signal as a function of pressure. As shown in the diagram, trace 310 shows the plethysmogram that corresponds to the pneumatic pressures applied to the patient's finger. In this case, as can be seen on trace 310, at a low end 315 of the pressure (e.g., approximately 50-80 mmHg), pulsatility 317 is low relative to the maximum pulsatility 318, which may indicate that finger cuff 202 is properly fitted (FIG. 3C). Similarly, with reference to FIG. 3B, the diagram illustrates the finger blood volume, and particularly, another example of the changing pleth signal as a function of pressure. As shown, a trace 320 shows a finger blood volume at every pneumatic pressure level applied to the patient's finger. As can be seen on trace 320, at a low end 325 of the pressure (e.g., approximately 30-60 mmHg), increase in DC pleth is gradual, whereas in the mid-range 327 of the pressure (e.g. approximately 80-120 mm Hg) the increase in DC Pleth is noticeably higher. The transition from gradual increase at low end 325 to rapid increase at mid-range 327 occurs between the low end of the pressure (approximately 30-60 mmHg) and the mid-range of the pressure (approximately 80-120 mmHg), which again may indicate that finger cuff 202 is properly fitted.

In contrast, referring to trace 410 of FIG. 4A, at a low end 415 of the pressure (e.g., approximately 30-60 mmHg), pulsatility 417, for example AC Pleth, is high compared to the peak pulsatility 418, which may indicate that finger cuff 202 is too tight (FIG. 4C). Similarly, with reference to FIG. 4B, the diagram illustrates the blood volume, and particularly, the DC pleth signal as a function of pressure. As shown, a trace 420 shows a finger blood volume at every pneumatic pressure level applied to the patient's finger. As can be seen on trace 420 of FIG. 4B, the increase in DC Pleth with pressure is roughly constant, that is, the trace 420 does not contain separate regions of low and rapid increase, in contrast to FIG. 3B, which again may indicate that finger cuff 202 is too tight.

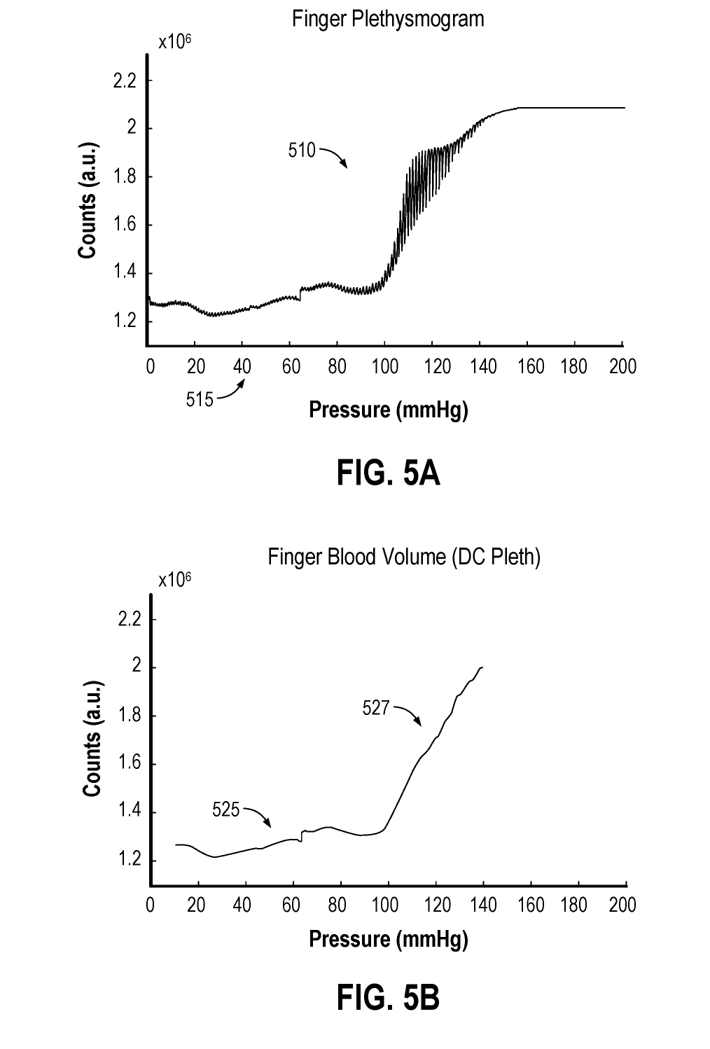

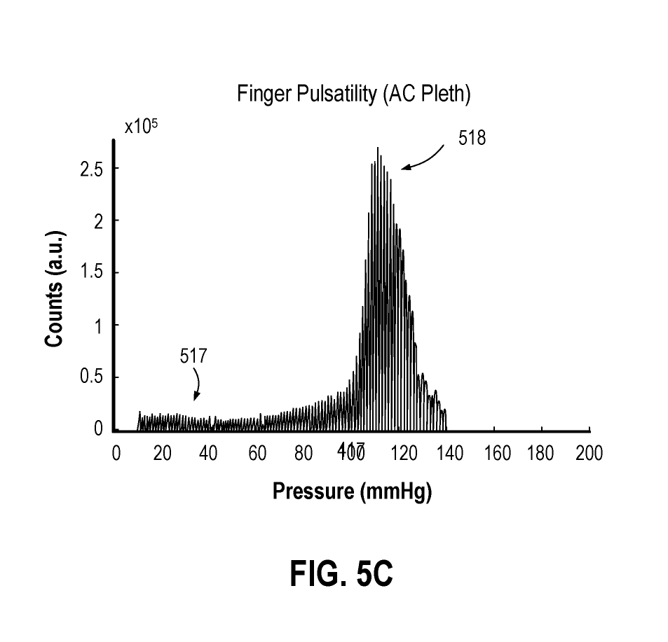

In another embodiment of the invention, referring to trace 510 in FIG. 5A, at the low end 515 of the pressure (e.g. approximately 30-60 mmHg), the pulsatility 517 is low compared to peak pulsatility 518, and furthermore, the pulsatility remains low well into the mid-range of the pressure (e.g. above 80 mm Hg), which may indicate the finger cuff 202 is too loose (FIG. 5C). Similarly, with reference to 5B, a region 525 of gradual increase in DC Pleth as a function of pressure and a separate region 527 of rapid increase in DC Pleth as a function of pressure are both present, similar to FIG. 3B. Unlike FIG. 3B, the transition from gradual increase of region 525 to rapid increase of region 527 occurs at a higher pressure (approximately 100 mm Hg), which may indicate the finger cuff 202 is too loose.

While FIGS. 3A-3C, 4A-4C, and 5A-5C illustrate a gradual pressure ramp response for the observations of the plethysmogram change, in some embodiments, a stepped increase (or “staircase”) and/or a large step response may be used for the observations of the plethysmogram change as a function of pressure.

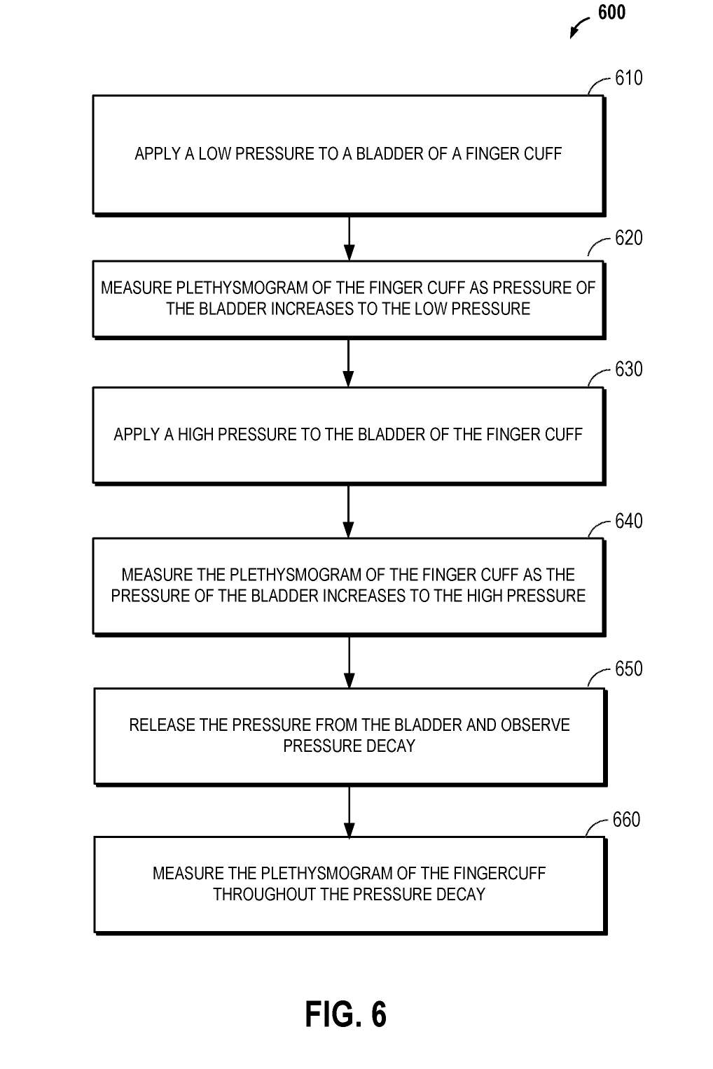

FIG. 6 is a flow diagram of a method for measuring the pulsatility of a finger cuff according to embodiments of the invention. Process 600 may be performed by processing logic that includes hardware (e.g. circuitry, dedicated logic, etc.), software (e.g., embodied on a non-transitory computer readable medium), or a combination thereof. For example, process 600 may be performed by pressure generating and regulating system 220, control circuitry 230, or a combination thereof.

Referring to FIG. 6, at block 610, the processing logic applies a low pressure (e.g., 20-40 mmHg) to a bladder (e.g., inflatable bladder 212) of a finger cuff (e.g., finger cuff 202). At block 620, the processing logic measures plethysmogram of the finger cuff as pressure of the bladder increases to the low pressure. At block 630, the processing logic applies a high pressure (e.g., 200 mmHg) to the bladder of the finger cuff. At block 640, the processing logic measures the plethysmogram, observing both the finger blood volume, or DC pleth, and the finger pulsatility, or AC pleth, of the finger cuff as the pressure of the bladder increases to the high pressure. At block 650, the processing logic releases the pressure from the bladder and observes pressure decay. At block 660, the processing logic measures the plethysmogram, observing both the finger blood volume, or DC pleth, and the finger pulsatility, or AC pleth, of the finger cuff throughout the pressure decay.

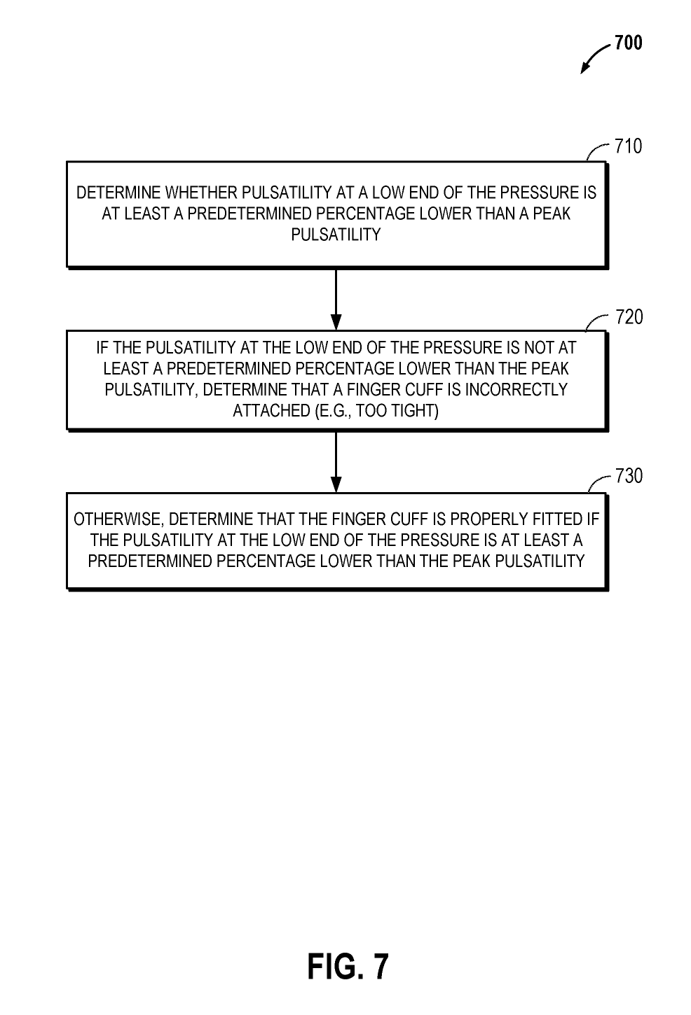

FIG. 7 is a flow diagram of a method for determining whether a finger cuff is properly fitted on a patient's finger according to embodiments of the invention. Process 700 may be performed by processing logic that includes hardware (e.g. circuitry, dedicated logic, etc.), software (e.g., embodied on a non-transitory computer readable medium), or a combination thereof. For example, process 700 may be performed by pressure generating and regulating system 220, control circuitry 230, or a combination thereof.

Referring to FIG. 7, at block 710, the processing logic determines whether pulsatility of a low end of the pressure (e.g., low end 315 of FIG. 3A) is at least a predetermined percentage lower than a peak pulsatility (or AC pleth level). At block 720, if the pulsatility at the low end of the pressure is not at least a predetermined percentage lower than the peak pulsatility (e.g., as shown in pulsatility 417 and 418 and in pulsatility 517 and 518), the processing logic determines that the finger cuff (e.g., finger cuff 202) is incorrectly attached (e.g., too tight, rotated or offset from the phalanx center). In this case, an operator (e.g., healthcare provider) may be instructed, via patient monitoring device 130 for example, to remove and reapply (e.g., loosen) the finger cuff. In some embodiments, if the incorrect attachment of the finger cuff occurs for a predetermined number of times (e.g., three times), the operator may be instructed to select a larger finger cuff. Otherwise, at block 730, the processing logic determines that the finger cuff is properly fitted if the pulsatility at the low end of the pressure is at least a predetermined percentage lower than the peak pulsatility (e.g., as shown in pulsatility 317 and 318).

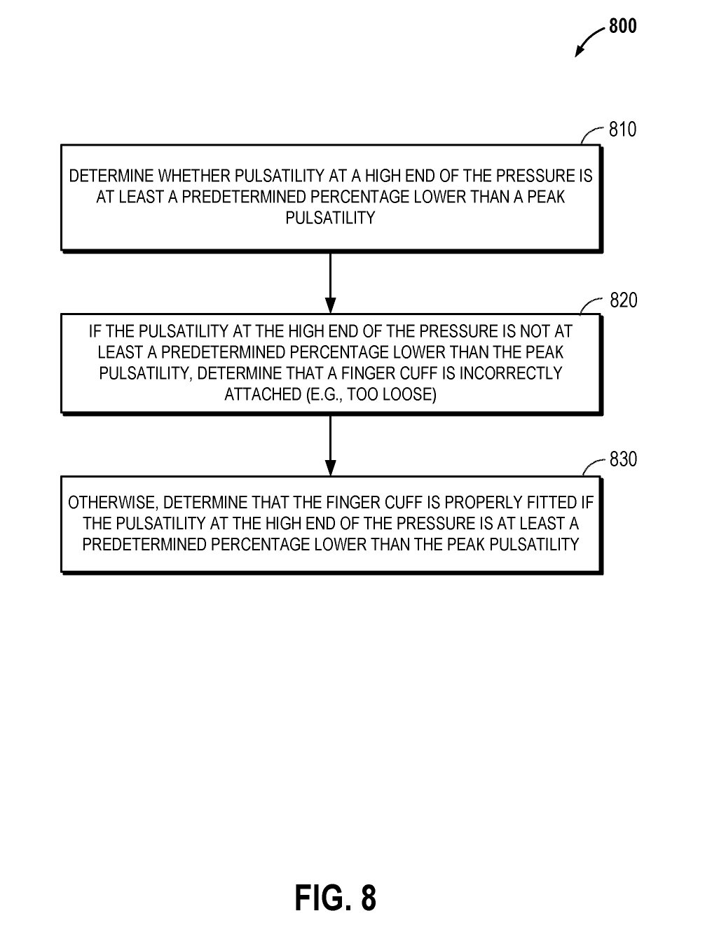

FIG. 8 is a flow diagram of another method for determining whether a finger cuff is properly fitted on a patient's finger according to embodiments of the invention. Process 800 may be performed by processing logic that includes hardware (e.g. circuitry, dedicated logic, etc.), software (e.g., embodied on a non-transitory computer readable medium), or a combination thereof. For example, process 800 may be performed by pressure generating and regulating system 220, control circuitry 230, or a combination thereof.

Referring to FIG. 8, at block 810, the processing logic determines whether pulsatility of a high end of the pressure is at least a predetermined percentage lower than a peak pulsatility (or pleth level). At block 820, if the pulsatility at the high end of the pressure is not at least a predetermined percentage lower than the peak pulsatility, the processing logic determines that the finger cuff (e.g., finger cuff 202) is incorrectly attached (e.g., too loose, rotated or offset from the phalanx center). In this case, the operator may be instructed, via patient monitoring device 130 for example, to remove and reapply the finger cuff (e.g., to reapply the finger cuff more tightly). In some embodiments, if the incorrect attachment of the finger cuff occurs for a predetermined number of times (e.g., three times), the operator may be instructed to select a smaller finger cuff. Otherwise, at block 830, the processing logic determines that the finger cuff is properly fitted if the pulsatility at the high end of the pressure is at least a predetermined percentage lower than the peak pulsatility.

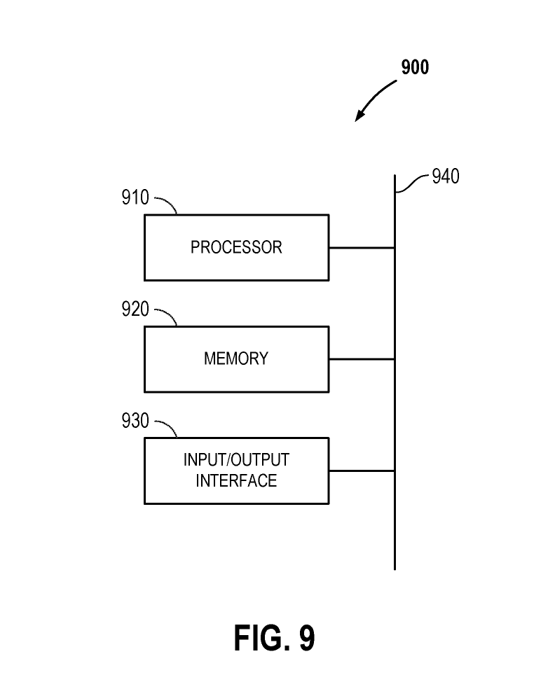

Referring to FIG. 9, a block diagram illustrating example control circuitry 230 is shown. It should be appreciated that FIG. 9 illustrates a non-limiting example of a control circuitry 230 implementation. Other implementations of the control circuitry 230 not shown in FIG. 9 are also possible. The control circuitry 230 may comprise a processor 910, a memory 920, and an input/output interface 930 connected with a bus 940. Under the control of the processor 910, data may be received from an external source through the input/output interface 930 and stored in the memory 920, and/or may be transmitted from the memory 920 to an external destination through the input/output interface 930. The processor 910 may process, add, remove, change, or otherwise manipulate data stored in the memory 920. Further, code may be stored in the memory 920. The code, when executed by the processor 910, may cause the processor 910 to perform operations relating to data manipulation and/or transmission and/or any other possible operations.

It should be appreciated that aspects of the invention previously described may be implemented in conjunction with the execution of instructions by processors, circuitry, controllers, control circuitry, etc. As an example, control circuity may operate under the control of a program, algorithm, routine, or the execution of instructions to execute methods or processes in accordance with embodiments of the invention previously described. For example, such a program may be implemented in firmware or software (e.g. stored in memory and/or other locations) and may be implemented by processors, control circuitry, and/or other circuitry, these terms being utilized interchangeably. Further, it should be appreciated that the terms processor, microprocessor, circuitry, control circuitry, circuit board, controller, microcontroller, etc., refer to any type of logic or circuitry capable of executing logic, commands, instructions, software, firmware, functionality, etc., which may be utilized to execute embodiments of the invention.

The various illustrative logical blocks, processors, modules, and circuitry described in connection with the embodiments disclosed herein may be implemented or performed with a general purpose processor, a specialized processor, circuitry, a microcontroller, a digital signal processor (DSP), an application specific integrated circuit (ASIC), a field programmable gate array (FPGA) or other programmable logic device, discrete gate or transistor logic, discrete hardware components, or any combination thereof designed to perform the functions described herein. A processor may be a microprocessor or any conventional processor, controller, microcontroller, circuitry, or state machine. A processor may also be implemented as a combination of computing devices, e.g., a combination of a DSP and a microprocessor, a plurality of microprocessors, one or more microprocessors in conjunction with a DSP core, or any other such configuration.

The steps of a method or algorithm described in connection with the embodiments disclosed herein may be embodied directly in hardware, in a software module/firmware executed by a processor, or any combination thereof. A software module may reside in RAM memory, flash memory, ROM memory, EPROM memory, EEPROM memory, registers, hard disk, a removable disk, a CD-ROM, or any other form of storage medium known in the art. An exemplary storage medium is coupled to the processor such the processor can read information from, and write information to, the storage medium. In the alternative, the storage medium may be integral to the processor.

The previous description of the disclosed embodiments is provided to enable any person skilled in the art to make or use the present invention. Various modifications to these embodiments will be readily apparent to those skilled in the art, and the generic principles defined herein may be applied to other embodiments without departing from the spirit or scope of the invention. Thus, the present invention is not intended to be limited to the embodiments shown herein but is to be accorded the widest scope consistent with the principles and novel features disclosed herein.

What is claimed is:

1. A system to monitor a finger cuff connectable to a patient's finger to be used in measuring the patient's blood pressure by a blood pressure measurement system utilizing the volume clamp method and to measure the plethysmogram of the finger cuff, the system comprising:
the finger cuff including an enclosing portion that encloses a patient's finger, the enclosing portion including a bladder and a light emitting diode (LED) and photodiode (PD) pair; and
a processor configured to:
command applying pneumatic pressure to the bladder of the finger cuff from a low pressure to a high pressure;
measure the plethysmogram of the finger cuff as the pressure increases from the low pressure to the high pressure; and
determine fitness of the finger cuff on the patient's finger based on the measured plethysmogram, wherein, when the finger cuff is placed around the patient's finger, the bladder and the LED-PD pair aid in measuring the plethysmogram.

2. The system of claim 1, wherein the processor is further to command releasing the pressure from the bladder and to observe pressure decay, and to measure the plethysmogram of the finger cuff throughout the pressure decay.

3. The system of claim 1, wherein determining the fitness of the finger cuff on the patient's finger comprises determining whether the finger cuff is loose, properly fitted, or too tight.

4. The system of claim 3, wherein determining whether the finger cuff is loose comprises: determining whether the pulsatility at a high end of the pressure is at least a predetermined percentage lower than a peak pulsatility.

5. The system of claim 4, wherein the processor is further to instruct an operator to reapply the finger cuff more tightly if the pulsatility at the high end of the pressure is not at least the predetermined percentage lower than the peak pulsatility.

6. The system of claim 3, wherein determining whether the finger cuff is properly fitted comprises: determining whether the pulsatility at a low end of the pressure is low.

7. The system of claim 3, wherein determining whether the finger cuff is too tight comprises: determining whether the pulsatility at a low end of the pressure is at least a predetermined percentage lower than a peak pulsatility.

8. The system of claim 7, wherein the processor is further to instruct an operator to loosen the finger cuff if the pulsatility at the low end of the pressure is not at least the predetermined percentage lower than the peak pulsatility.

9. The system of claim 6, wherein after the finger cuff is determined to be properly fitted, the finger cuff is used in measuring the patient's blood pressure by the blood pressure measurement system utilizing the volume clamp method.

10. The system of claim 1, wherein the processor is further configured to:
determine whether blood volume of the patient's finger measured at the end of recovery time has returned to an initial value measured at the low pressure; and
in response to determining that the blood volume of the patient's finger measured at the end of the recovery time has not returned to the initial value:
determine that the patient's perfusion is too low for the blood pressure measurement system to operate properly, and
instruct an operator to increase the patient's perfusion by warming the hand or to select a different pressure monitoring technology.

11. A method to monitor a finger cuff connectable to a patient's finger to be used in measuring the patient's blood pressure by a blood pressure measurement system utilizing the volume clamp method and to measure the plethysmogram of the finger cuff, the finger cuff including an enclosing portion that encloses a patient's finger, the enclosing portion including a bladder and a light emitting diode (LED) and photodiode (PD) pair, the method comprising:
applying pneumatic pressure to the bladder of the finger cuff from a low pressure to a high pressure;
measuring the plethysmogram of the finger cuff as the pressure increases from the low pressure to the high pressure; and
determining fitness of the finger cuff on the patient's finger based on the measured plethysmogram, wherein, when the finger cuff is placed around the patient's finger, the bladder and the LED-PD pair aid in measuring the plethysmogram.

12. The method of claim 11, further comprising: releasing the pressure from the bladder and observing pressure decay, and measuring the plethysmogram of the finger cuff throughout the pressure decay.

13. The method of claim 11, wherein determining the fitness of the finger cuff on the patient's finger comprises determining whether the finger cuff is loose, properly fitted, or too tight.

14. The method of claim 13, wherein determining whether the finger cuff is loose comprises: determining whether the pulsatility at a high end of the pressure is at least a predetermined percentage lower than a peak pulsatility.

15. The method of claim 14, further comprising: instructing an operator to reapply the finger cuff more tightly if the pulsatility at the high end of the pressure is not at least the predetermined percentage lower than the peak pulsatility.

16. The method of claim 13, wherein determining whether the finger cuff is properly fitted comprises: determining whether the pulsatility at a low end of the pressure is low.

17. The method of claim 13, wherein determining whether the finger cuff is too tight comprises: determining whether the pulsatility at a low end of the pressure is at least a predetermined percentage lower than a peak pulsatility.

18. The method of claim 17, further comprising: instructing an operator to loosen the finger cuff if the pulsatility at the low end of the pressure is not at least the predetermined percentage lower than the peak pulsatility.

19. The method of claim 16, wherein after the finger cuff is determined to be properly fitted, the finger cuff is used in measuring the patient's blood pressure by the blood pressure measurement system utilizing the volume clamp method.

20. The method of claim 11, further comprising:
determining whether blood volume of the patient's finger measured at the end of recovery time has returned to an initial value measured at the low pressure;
in response to determining that the blood volume of the patient's finger measured at the end of the recovery time has not returned to the initial value:
determining that the patient's perfusion is too low for the blood pressure measurement system to operate properly, and
instructing an operator to increase the patient's perfusion by warming the hand or to select a different pressure monitoring technology.
